(19) United States
(12) Patent Application Publication
(10) Pub. No.: US 2022/0401330 A1
(43) Pub. Date: Dec. 22, 2022

(54) METHOD FOR THE DECOLORIZATION OF KERATIN MATERIAL THAT HAS BEEN DYED BY APPLYING A PIGMENT

(71) Applicant: Henkel AG & Co. KGaA, Duesseldorf (DE)

(72) Inventors: Constanze KRUCK, Grevenbroich (DE); Sandra HILBIG, Bochum (DE); Melanie MOCH, Dormagen (DE); Susanne DICKHOF, Viersen (DE); Daniela KESSLER-BECKER, Leverkusen (DE)

(73) Assignee: Henkel AG & Co. KGaA, Duesseldorf (DE)

(21) Appl. No.: 17/780,014

(22) PCT Filed: Sep. 23, 2020
(86) PCT No.: PCT/EP2020/076468

§ 371 Date: May 25, 2022

(30) Foreign Application Priority Data
Nov. 26, 2019 (DE) 10 2019 218 227.5

(51) Int. Cl. A61K 8/46; A61K 8/896; A61K 8/36; A61K 8/42; A61Q 5/06; A61Q 5/08

(52) U.S. Cl. CPC A61K 8/463; A61K 8/896; A61K 8/36; A61K 8/42; A61Q 5/06; A61Q 5/08; A61K 2800/43; A61K 2800/884

(57) ABSTRACT

Processes and a multi-component packaging unit (kit-of-parts) are provided for decolorizing keratinous material which has been colored by the application of at least one pigment. A process includes applying to the keratinous material a decolorizing agent comprising at least one anionic surfactant, and having a pH value of 1.0 to 3.5, and rinsing the decolorization agent from the keratinous material after a contact time.

### CROSS-REFERENCE TO RELATED APPLICATION

This application is a U.S. National-Stage entry under 35 U.S.C. § 371 based on International Application No. PCT/EP2020/076468, filed Sep. 23, 2020, which was published under PCT Article 21(2) and which claims priority to German Application No. 102019218227.5, filed Nov. 26, 2019, which are all hereby incorporated in their entirety by reference.

### TECHNICAL FIELD

The present application is in the field of cosmetics and relates to a process for decolorizing keratin material which has been colored by the application of at least one pigment. The decolorant applied in this process is exemplified by its content of at least one anionic surfactant and a pH value which is in the range from 1.0 to 3.5. The decolorizing agent is applied to the dyed keratin material and rinsed off again after a reaction time.

A second object of the present application is a process for dyeing and later decolorizing keratin material, in which first a dyeing agent comprising at least one amino-functionalized silicone polymer and a pigment is applied to the keratin, and in the subsequent steps the decolorization is carried out by applying the decolorizing agent described above.

A third object of the present application is a multi-component packaging unit which comprises the coloring agent and the decolorizing agent in separately prepared containers.

### BACKGROUND

The change in shape and color of keratin fibers, especially hair, is an important area of modern cosmetics. To change the hair color, the expert knows various coloring systems depending on coloring requirements. Oxidation dyes are usually used for permanent, intensive dyeings with good fastness properties and good grey coverage. Such dyes usually contain oxidation dye precursors, so-called developer components and coupler components, which form the actual dyes with one another under the influence of oxidizing agents, such as hydrogen peroxide. Oxidation dyes are exemplified by very long-lasting dyeing results.

When direct dyes are used, ready-made dyes diffuse from the colorant into the hair fiber. Compared to oxidative hair dyeing, the dyeings obtained with direct dyes have a shorter shelf life and quicker wash ability. Dyes with direct dyes usually remain on the hair for a period of between 5 and 20 washes.

The use of color pigments is known for short-term color changes on the hair and/or skin. Color pigments are generally understood to be insoluble, coloring substances. These are present undissolved in the dye formulation in the form of small particles and are only deposited from the outside on the hair fibers and/or the skin surface. Therefore, they can usually be removed again without residue by a few washes with detergents comprising surfactants. Various products of this type are available on the market under the name hair mascara.

If the user wants particularly long-lasting dyeings, the use of oxidative dyes has so far been his only option. However, despite numerous optimization attempts, an unpleasant ammonia or amine odor cannot be completely avoided in oxidative hair dyeing. The hair damage still associated with the use of oxidative dyes also has a negative effect on the user's hair. A continuing challenge is therefore the search for alternative, high-performance dyeing processes. One possible alternative coloring system that has recently come increasingly into focus is based on the use of colored pigments.

Coloring with pigments offers several significant advantages. Since the pigments only attach themselves to the keratin materials, especially the hair fibers, from the outside, the damage associated with the dyeing process is particularly low.

### BRIEF SUMMARY

A process is provided for decolorizing keratinous material which has been colored by the application of at least one pigment. The process includes applying to the keratinous material a decolorizing agent comprising at least one anionic surfactant, and having a pH value of 1.0 to 3.5, and rinsing the decolorization agent from the keratinous material after a contact time.

A process is provided for dyeing and later decolorizing human hair. The process includes (1) applying a colorant to the hair, the colorant comprising at least one amino-functionalized silicone polymer and at least one pigment, (2) allowing the dye to act on the hair, (3) rinsing the dye from the hair, (4) applying a decolorizing agent to the hair, the decolorizing agent comprising at least one anionic surfactant and having a pH value of 1.0 to 3.5, (5) allowing the decolorizing agent to act on the hair, and (6) rinsing the decolorizing agent out of the hair.

A multi-component packaging unit (kit-of-parts) is provided for staining and decolorizing keratin material. The kit of parts includes, separately packaged: a first container comprising a colorant, the colorant comprising at least one amino-functionalized silicone polymer and at least one pigment, and a second container comprising a decolorizing agent, the decolorizing agent comprising at least one anionic surfactant and having a pH value of 1.0 to 3.5.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

In recent work, the problem of low durability of this staining system has been addressed. In this context, it was found that the wash fastness of the color results obtained with pigments could be greatly improved by combining the pigments with certain amino-functionalized silicone polymers.

By using a suitable decolorizing agent, it is possible to remove these colorations without affecting the user's original hair color. In this way, the user has the option of returning to his original hair color immediately and without much effort. Especially for those consumers who do not want to recolor their hair regularly, this coloring process is therefore particularly attractive.

A well-suited decolorizing agent should be able to remove the colored film formed on the surface of the keratin material by the application of pigments or pigments and amino silicones as far as possible without leaving any residue. If this colored film is only partially or incompletely removed, undesirable color shifts or a blotchy color result are the consequence, which are perceived as highly unattractive by the user.

To increase user comfort, a decolorizing agent should remove the colored films within the shortest possible period of time, and the keratin material or hair should not be damaged by application of the decolorizing agent.

The object of the present disclosure was to provide a decolorizing agent for decolorizing dyed keratinous fibers previously dyed by application of at least one pigment, or of at least one amino-functionalized silicone polymer and a pigment. Here, the decolorization should be as complete as possible so that the keratin material can ideally be restored to its original color state. Furthermore, the decolorization should be long-lasting and uniform, and the decolorized keratin fibers should suffer neither shifts in nuance nor irregularities in the color result. In addition, the keratin material should be damaged as little as possible by the decolorizing agent.

Surprisingly, it has now been found that this task can be fully solved if keratinous material previously colored with at least one amino silicone and with at least one pigment is treated with a decolorizing agent which comprises at least one anionic surfactant and has a pH in the range from 1.0 to 3.5.

A first object of the present disclosure is a process for decolorizing keratin material which has been colored by application of at least one pigment, wherein a decolorizing agent which is

(a) comprises at least one anionic surfactant, and

(b) has a pH value of 1.0 to 3.5,

is applied to the dyed keratin material and rinsed off again after a contact time.

Work leading to the present disclosure has shown that keratin fibers, particularly hair, could be intensely colored by the application of pigments. Particularly intensive staining results were obtained when staining was carried out with a combination of pigment and amino silicone. The pigment or the mixture of pigment and amino silicone was deposited in the form of a colored film on the surface of the keratin fibers. Furthermore, it has been shown that these colorations could be completely decolorized again within a short application period by using the previously described decolorizing agent, without damaging the hair.

### Decolorization of Keratinous Material

Keratinous material includes hair, skin, nails (such as fingernails and/or toenails). Wool, furs and feathers also fall under the definition of keratinous material.

Preferably, keratinous material is understood to be human hair, human skin and human nails, especially fingernails and toenails. Keratinous material is understood to be human hair in particular.

The term “agent for decolorization” is understood in the context of the present disclosure to mean that a coloration produced on the keratin material by application of at least one pigment, or at least one amino-functionalized silicone polymer and a pigment, can be removed again. In this dyeing process, the keratin material or keratin fiber is coated with a dyed film formed from the pigment or pigment and amino silicone. As contemplated herein, the application of the decolorizing agent takes place after the application of the colorant and is able to remove this colored film from the keratin material again.

Characteristic of the process as contemplated herein is the application of the decolorizing agent to keratin material which has previously been colored by application of at least one pigment, or at least one pigment and amino silicone.

### Amino Functionalized Silicone Polymer in the Colorant

The decolorizing agent used in the process as contemplated herein showed a particularly strong effect when a combination of pigments with amino silicones was used in the preceding coloration of the keratin material or keratin fibers.

In the context of a very particularly preferred embodiment, a process as contemplated herein is therefore wherein the decolorizing agent is applied to keratin material which has been colored by application of at least one amino-functionalized silicone polymer and at least one pigment.

The amino-functionalized silicone polymer may alternatively be referred to as amino silicone or amodimethicone.

Silicone polymers are generally macromolecules with a molecular weight of at least 500 g/mol, preferably at least 1000 g/mol, more preferably at least 2500 g/mol, particularly preferably at least 5000 g/mol, which comprise repeating organic units.

The maximum molecular weight of the silicone polymer depends on the degree of polymerization (number of polymerized monomers) and the batch size and is also partly determined by the polymerization method. For the purposes of the present disclosure, it is preferred if the maximum molecular weight of the silicone polymer is not more than 10⁷ g/mol, preferably not more than 10⁶ g/mol, and particularly preferably not more than 10⁵ g/mol.

The silicone polymers comprise many Si—O repeating units, and the Si atoms may carry organic radicals such as alkyl groups or substituted alkyl groups. Alternatively, a silicone polymer is therefore also referred to as polydimethylsiloxane.

Corresponding to the high molecular weight of silicone polymers, these are based on more than 10 Si—O repeat units, preferably more than 50 Si—O repeat units, and more preferably more than 100 Si—O repeat units, most preferably more than 500 Si—O repeat units.

An amino-functionalized silicone polymer is understood to be a functionalized silicone that carries at least one structural unit with an amino group. Preferably, the amino-functionalized silicone polymer carries multiple structural units, each having at least one amino group. An amino group is understood to mean a primary amino group, a secondary amino group and a tertiary amino group. All these amino groups can be protonated in the acidic environment and are then present in their cationic form.

In principle, good dyeing performance could be achieved with amino-functionalized silicone polymers if they carry at least one primary, at least one secondary and/or at least one tertiary amino group. However, intense colorations with the best wash fastness were obtained when an amino-functionalized silicone polymer comprising at least one secondary amino group was used in the agent.

In a very particularly preferred embodiment, a process as contemplated herein is wherein the decolorizing agent is applied to keratin material which has been colored by application of at least one amino-functionalized silicone polymer having at least one secondary amino group.

The secondary amino group(s) may be located at various positions on the amino-functionalized silicone polymer. Particularly good color results were obtained when an amino-functionalized silicone polymer was used which has at least one, preferably several, structural units of the formula (Si-Amino).

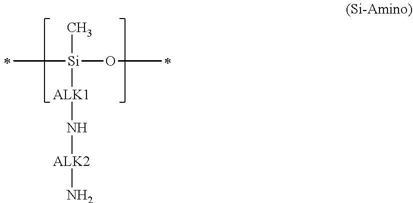

In the structural units of the formula (Si amino), the abbreviations ALK1 and ALK2 independently stand for a linear or branched, bivalent C₁-C₂₀ alkylene group.

In another very particularly preferred embodiment, a process as contemplated herein is wherein the decolorizing agent is applied to keratin material which has been colored by application of at least one amino-functionalized silicone polymer comprising at least one structural unit of the formula (Si-Amino),

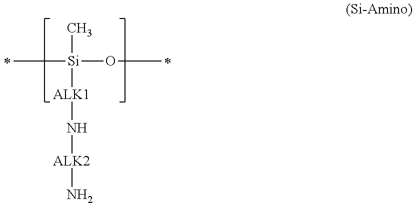

where

ALK1 and ALK2 independently represent a linear or branched C₁-C₂₀ divalent alkylene group.

The positions marked with an asterisk (*) indicate the bond to further structural units of the silicone polymer. For example, the silicon atom adjacent to the star may be bonded to another oxygen atom, and the oxygen atom adjacent to the star may be bonded to another silicon atom or even to a C₁-C₆ alkyl group.

A bivalent C₁-C₂₀ alkylene group can alternatively be referred to as a divalent or divalent C₁-C₂₀ alkylene group, by which is meant that each ALK1 or AK2 grouping can form two bonds.

In the case of ALK1, one bond occurs from the silicon atom to the ALK1 grouping, and the second bond is between ALK1 and the secondary amino group.

In the case of ALK2, one bond is from the secondary amino group to the ALK2 grouping, and the second bond is between ALK2 and the primary amino group.

Examples of a linear bivalent C₁-C₂₀ alkylene group include the methylene group (—CH₂—), the ethylene group (—CH₂—CH₂—), the propylene group (—CH₂—CH₂—CH₂—), and the butylene group (—CH₂—CH₂—CH₂—CH₂—). The propylene group (—CH₂—CH₂—CH₂—) is particularly preferred. From a chain length of 3 C atoms, bivalent alkylene groups can also be branched. Examples of branched divalent, bivalent C₃-C₂₀ alkylene groups are (—CH₂—CH(CH₃)—) and (—CH₂—CH(CH₃)—CH₂—).

In another particularly preferred embodiment, the structural units of the formula (Si amino) represent repeat units in the amino-functionalized silicone polymer, such that the silicone polymer comprises multiple structural units of the formula (Si amino).

Particularly well-suited amino-functionalized silicone polymers with at least one secondary amino group are listed below.

Dyeings with the very best wash fastnesses could be obtained if, during the preceding dyeing, at least one agent comprising at least one amino-functionalized silicone polymer comprising structural units of formula (Si-I) and formula (Si-II) was applied to the keratinous material

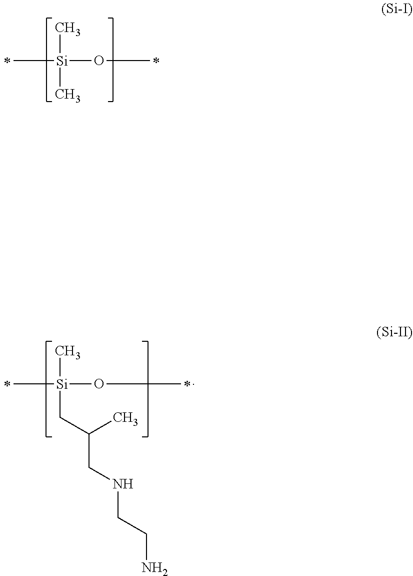

In a further explicitly very particularly preferred embodiment, a process as contemplated herein is wherein the decolorizing agent is applied to keratin material which has been colored by application of at least one amino-functionalized silicone polymer which comprises structural units of the formula (Si-I) and of the formula (Si-II)

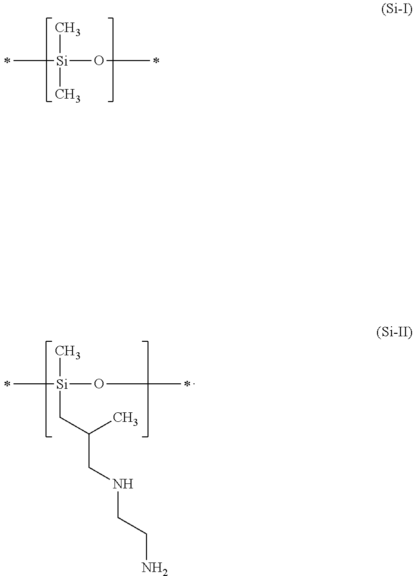

A corresponding amino functionalized silicone polymer with the structural units (Si-I) and (Si-II) is, for example, the commercial product DC 2-8566 or Dowsil 2-8566 Amino Fluid, which is commercially distributed by the Dow Chemical Company and bears the designation “Siloxanes and Silicones, 3-1(2-aminoethyl]aminol-2-methylpropyl Me, Di-Me-Siloxane” and the CAS number 106842-44-8. Another particularly preferred commercial product is Dowsil AP-8658 Amino Fluid, which is also sold commercially by the Dow Chemical Company.

In another preferred embodiment, the decolorizing agent may also be applied to keratin material previously colored by the application of a colorant comprising at least one amino-functional silicone polymer of the formula of formula (Si-III),

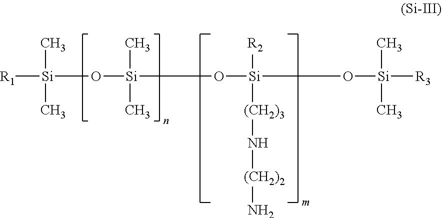

where
m and n mean numbers chosen so that the sum (n+m) is in the range 1 to 1000,
n is a number in the range 0 to 999 and m is a number in the range 1 to 1000,
R1, R2 and R3, which are the same or different, denote a hydroxy group or a C1-4 alkoxy group,
wherein at least one of R1 to R3 represents a hydroxy group;

Further processes preferred as contemplated herein are exemplified by the prior application of a colorant to the keratinous material, the colorant comprising at least amino-functional silicone polymer of the formula of formula (Si-IV),

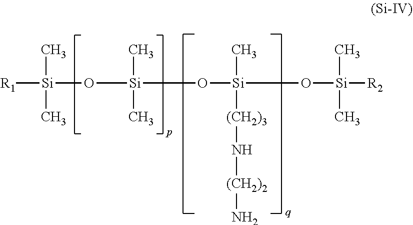

located in the
p and q mean numbers chosen so that the sum (p+q) is in the range 1 to 1000,
p is a number in the range 0 to 999 and q is a number in the range 1 to 1000,
R1 and R2, which are different, denote a hydroxy group or a C1-4 alkoxy group, at least one of R1 to R2 denoting a hydroxy group.

The silicones of the formulas (Si-III) and (Si-IV) differ in the grouping at the Si atom, which carries the nitrogen-comprising group: In formula (Si-III), R2 represents a hydroxy group or a C₁-4 alkoxy group, while the radical in formula (Si-IV) is a methyl group. The individual Si groupings, which are marked with the indices m and n or p and q, do not have to be present as blocks; rather, the individual units can also be present in a statistically distributed manner, i.e., in the formulas (Si-III) and (Si-IV), not every R1-Si(CH₃)₂ group is necessarily bonded to an —[O—Si(CH₃)₂] grouping.

Processes as contemplated herein in which a colorant comprising at least one amino-functional silicone polymer of the formula (Si-V) is applied to the keratin fibers have also proved to be particularly effective in producing intense color results.

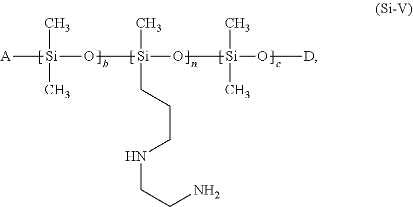

located in the
A represents a group —OH, —O—Si(CH₃)₃, —O—Si(CH₃)₂OH, —O—Si(CH₃)₂OCH₃,
D represents a group —H, —Si(CH₃)₃, —Si(CH₃)₂OH, —Si(CH₃)₂OCH₃,
b, n and c stand for integers between 0 and 1000,
with the specifications
n>0 and b+c>0
at least one of the conditions A=-OH or D=-H is fulfilled.

In the above formula (Si-V), the individual siloxane units are statistically distributed with the indices b, c and n, i.e., they do not necessarily have to be block copolymers.

The previously applied colorant may further comprise one or more different amino-functionalized silicone polymers represented by the formula (Si-VI)

M(RₐQ_{b}SiO_{(4-a-b)/2)x}(R_{c}SiO_{(4-c)/2)y}M (Si-VI)

in which formula above R is a hydrocarbon or a hydrocarbon radical having from 1 to about 6 carbon atoms, Q is a polar radical of the general formula —R¹HZ wherein R¹ is a divalent linking group bonded to hydrogen and the radical Z composed of carbon and hydrogen atoms, carbon, hydrogen and oxygen atoms, or carbon, hydrogen and nitrogen atoms, and Z is an organic amino functional radical comprising at least one amino functional group; “a” takes values ranging from about 0 to about 2, “b” takes values ranging from about 1 to about 3, “a”+“b” is less than or equal to 3, and “c” is a number ranging from about 1 to about 3, and x is a number ranging from 1 to about 2,000, preferably from about 3 to about 50 and most preferably from about 3 to about 25, and y is a number in the range of from about 20 to about 10,000, preferably from about 125 to about 10,000 and most preferably from about 150 to about 1,000, and M is a suitable silicone end group as known in the prior art, preferably trimethylsiloxy. Non-limiting examples of radicals represented by R include alkyl radicals, such as methyl, ethyl, propyl, isopropyl, isopropyl, butyl, isobutyl, amyl, isoamyl, hexyl, isohexyl and the like; alkenyl radicals, such as vinyl, halovinyl, alkylvinyl, allyl, haloallyl, alkylallyl; cycloalkyl radicals, such as cyclobutyl, cyclopentyl, cyclohexyl and the like; phenyl radicals, benzyl radicals, halohydrocarbon radicals, such as 3-chloropropyl, 4-bromobutyl, 3,3,3-trifluoropropyl, chlorocyclohexyl, bromophenyl, chlorophenyl and the like, and sulfur-comprising radicals, such as mercaptoethyl, mercaptopropyl, mercaptohexyl, mercaptophenyl and the like; preferably R is an alkyl radical comprising from 1 to about 6 carbon atoms, and most preferably R is methyl. Examples of R¹ include methylene, ethylene, propylene, hexamethylene, decamethylene, —CH₂CH(CH₃)CH₂—, phenylene, naphthylene, —CH₂CH₂SCH₂CH₂—, —CH₂CH₂OCH₂—, —OCH₂CH₂—, —OCH₂CH₂CH₂—, —CH₂CH(CH₃)C(O)OCH₂—, —(CH₂)₃CC(O)OCH₂CH₂—, —C₆H₄C₆H₄—, —C₆H₄CH₂C₆H₄—; and —(CH₂)₃C(O)SCH₂CH₂—.
Z is an organic amino functional radical comprising at least one amino functional group. One possible formula for Z is NH(CH₂)_{z}NH₂, where z is 1 or more. Another possible formula for Z is —NH(CH₂)_{z}(CH₂)_{zz}NH, wherein both z and zz are independently 1 or more, this structure comprising diamino ring structures, such as piperazinyl. Z is most preferably an —NHCH₂CH₂NH₂ radical. Another possible formula for Z is —N(CH₂)_{z}(CH₂)_{zz}NX₂ or —NX₂, wherein each X of X₂ is independently selected from the group of hydrogen and alkyl groups having 1 to 12 carbon atoms, and zz is 0.
Q is most preferably a polar, amine-functional radical of the formula —CH₂CH₂CH₂NHCH₂CH₂NH 2. In the formulas, “a” takes values ranging from about 0 to about 2, “b” takes values ranging from about 2 to about 3, “a”+“b” is less than or equal to 3, and “c” is a number ranging from about 1 to about 3. The molar ratio of RₐQ_{b} SiO_{(4-a-b)>/2} units to R_{c}SiO_{(4-c)/2} units is in the range of about 1:2 to 1:65, preferably from about 1:5 to about 1:65 and most preferably by about 1:15 to about 1:20. If one or more silicones of the above formula are used, then the various variable substituents in the above formula may be different for the various silicone components present in the silicone mixture.

In a particularly preferred embodiment, a process as contemplated herein is exemplified by the prior application of a colorant to the keratinous material, said colorant comprising an amino-functional silicone polymer of formula (Si-VII)

R′ₐG₃₋ₐ-Si(OSiG₂)ₙ-(OSiG_{b}R′_{2-b})ₘ—O—SiG₃₋ₐ-R′ₐ (Si-VII),

wherein means:
G is —H, a phenyl group, —OH, —O—CH₃, —CH₃, —O—CH₂CH₃, —CH₂CH₃, —O—CH₂CH₂CH₃, —CH₂CH₂CH₃, —O—CH(CH₃)₂, —CH(CH₃)₂, —O—CH₂CH₂CH₂CH₃, —CH₂CH₂CH₂CH₃, —O—CH₂CH(CH₃)₂, —CH₂CH(CH₃)₂, —O—CH(CH₃)CH₂CH₃, —CH(CH₃)CH₂CH₃, —O—C(CH₃)₃, —C(CH₃)₃;
a stands for a number between 0 and 3, especially 0;
b stands for a number between 0 and 1, especially 1,
m and n are numbers whose sum (m+n) is between 1 and 2000, preferably between 50 and 150, where n preferably assumes values from 0 to 1999 and in particular from 49 to 149 and m preferably assumes values from 1 to 2000, in particular from 1 to 10,
R′ is a monovalent radical selected from
   -Q-N(R″)—CH₂—CH₂—N(R″)₂
   -Q-N(R″)₂
   -Q-N+(R″)₃A-
   -Q-N+H(R″)₂A-
   -Q-N+H₂(R″)A-
   -Q-N(R″)—CH₂—CH₂—N+R″H₂A-,
      where each Q is a chemical bond, —CH₂—, —CH₂—CH₂—, —CH₂CH₂CH₂—, —C(CH₃)₂—, —CH₂CH₂CH₂CH₂—, —CH₂C(CH₃)₂—, —CH(CH₃)CH₂CH₂—,
      R″ represents identical or different radicals selected from the group of —H, -phenyl, -benzyl, —CH₂—CH(CH₃)Ph, the C₁₋₂₀ alkyl radicals, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃, and A represents an anion preferably selected from chloride, bromide, iodide or methosulfate.

In the context of a further preferred embodiment, a process as contemplated herein is exemplified by the prior application of a colorant to the keratinous material, the colorant comprising at least one amino-functional silicone polymer of the formula (Si-VIIa),

(CH₃)₃Si—[O—Si(CH₃)₂]ₙ[OSi(CH₃)]ₘ—OSi(CH₃)₃ (Si-VIIa),

CH₂CH(CH₃)CH₂NH(CH₂)₂NH₂

wherein m and n are numbers whose sum (m+n) is between 1 and 2000, preferably between 50 and 150, n preferably assuming values from 0 to 1999 and in particular from 49 to 149, and m preferably assuming values from 1 to 2000, in particular from 1 to 10.

According to the INCI declaration, these silicones are called trimethylsilylamodimethicones.

In a further preferred embodiment, a process as contemplated herein is exemplified by the prior application of a colorant to the keratinous material, said colorant comprising at least one amino-functional silicone polymer of formula (Si-VIIb)

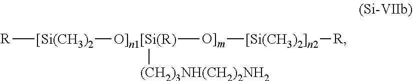

in which R represents —OH, —O—CH₃ or a —CH₃ group and m, n1 and n2 are numbers whose sum (m+n1+n2) is between 1 and 2000, preferably between 50 and 150, the sum (n1+n2) preferably assuming values from 0 to 1999 and in particular from 49 to 149 and m preferably assuming values from 1 to 2000, in particular from 1 to 10.

According to the INCI declaration, these amino-functionalized silicone polymers are called amodimethicones.

Regardless of which aminofunctional silicones are used, colorants as contemplated herein are preferred which contain an aminofunctional silicone polymer whose amine number is above 0.25 meq/g, preferably above 0.3 meq/g and in particular above 0.4 meq/g. The amine number represents the milliequivalents of amine per gram of the amino-functional silicone. It can be determined by titration and also expressed in the unit mg KOH/g.

Furthermore, colorants which included a special 4-morpholinomethyl-substituted silicone polymer are also suitable for use in the process as contemplated herein. This amino-functionalized silicone polymer comprises structural units of the formulae (SI-VIII) and of the formula (Si-IX)

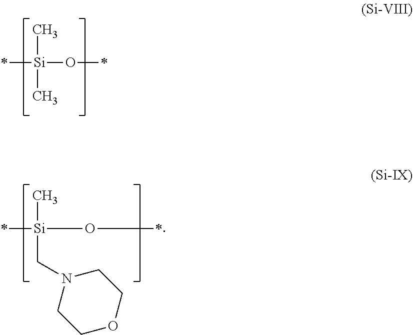

Corresponding 4-morpholinomethyl-substituted silicone polymers are described below.

A corresponding amino-functionalized silicone polymer is available under the name of Amodimethicone/Morpholinomethyl Silsesquioxane Copolymer is known and commercially available from Wacker in the form of the raw material Belsil ADM 8301 E.

As a 4-morpholinomethyl-substituted silicone, for example, a silicone can be used which has structural units of the formulae (Si-VIII), (Si-IX) and (Si-X)

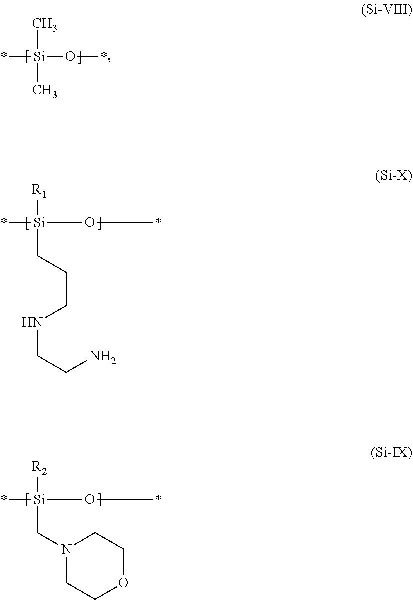

in which
R1 is —CH₃, —OH, —OCH₃, —O—CH₂CH₃, —O—CH₂CH₂CH₃, or —O—CH(CH₃)₂;
R2 is —CH₃, —OH, or —OCH₃.
Particularly preferred colorants contain at least one 4-morpholinomethyl-substituted silicone of the formula (Si-XI)

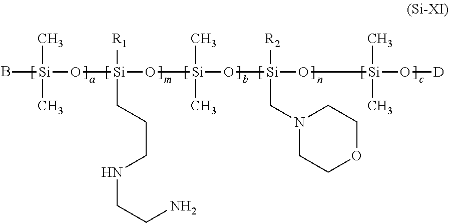

located in the
R1 is —CH₃, —OH, —OCH₃, —O—CH₂CH₃, —O—CH₂CH₂CH₃, or —O—CH(CH₃)₂;
R2 is —CH₃, —OH, or —OCH₃.
B represents a group —OH, —O—Si(CH₃)₃, —O—Si(CH₃)₂OH, —O—Si(CH₃)₂OCH₃,
D represents a group —H, —Si(CH₃)₃, —Si(CH₃)₂OH, —Si(CH₃)₂OCH₃,
a, b and c stand independently for integers between 0 and 1000, with the condition a+b+c>0
m and n independently of each other stand for integers between 1 and 1000

with the proviso that
at least one of the conditions B═—OH or D=-H is fulfilled,
the units a, b, c, m and n are distributed statistically or blockwise in the molecule.

Structural formula (Si-XI) is intended to illustrate that the siloxane groups n and m do not necessarily have to be directly bonded to a terminal grouping B or D, respectively. Rather, in preferred formulas (Si-VI) a>0 or b>0 and in particularly preferred formulas (Si-VI) a>0 and c>0, i.e., the terminal grouping B or D is preferably attached to a dimethylsiloxy grouping. Also, in formula (Si-VI), the siloxane units a, b, c, m and n are preferably statistically distributed.

The silicones used as contemplated herein represented by formula (Si-VI) can be trimethylsilyl-terminated (D or B=—Si(CH₃)₃), but they can also be dimethylsilylhydroxy-terminated on two sides or dimethylsilylhydroxy-terminated and dimethylsilylmethoxy-terminated on one side. Silicones particularly preferred in the context of the present disclosure are selected from silicones in which

B=—O—Si(CH₃)₂OH and D=—Si(CH₃)₃
B=—O—Si(CH₃)₂OH and D=—Si(CH₃)₂OH
B=—O—Si(CH₃)₂OH and D=—Si(CH₃)₂OCH₃
B=—O—Si(CH₃)₃ and D=—Si(CH₃)₂OH
B=—O—Si(CH₃)₂OCH₃ and D=—Si(CH₃)₂OH
to everyone. These silicones lead to exorbitant improvements in the hair properties of the hair treated with the agents of the present disclosure, and to a seriously improved protection in oxidative treatment.

In the agent used for the preceding coloration in the process as contemplated herein, one or more amino-functionalized silicone polymers may be present, for example, in a total amount of from 0.1 to 8.0 wt. %, preferably from 0.2 to 5.0 wt. %, more preferably from 0.3 to 3.0 wt. %, and most preferably from 0.4 to 2.5 wt. %. Here, the quantities are based on the total quantity of all amino silicones used, which is set in relation to the total weight of the colorant.

In this context, it was observed that the impression of greasy or oily hair also depends on the amount of amino silicones used. The higher the content of amino silicone in the colorant, the more negative was also the haptic impression. The use of the decolorizing agent as contemplated herein showed a particularly strong improvement in hair feel when a very high percentage by weight of amino silicones was used in the preceding application of the colorant.

In the context of a further particularly preferred embodiment, a process as contemplated herein is wherein the colorant comprises—based on the total weight of the agent—one or more amino-functionalized silicone polymers in a total amount of from 0.1 to 8.0 wt. %, preferably from 0.4 to 5.0 wt. %, more preferably from 0.8 to 3.0 wt. % and very particularly preferably from 1.0 to 3.5 wt. %.

### Pigments in the Colorant

In the process as contemplated herein, a decolorizing agent is applied to keratin material that has previously been colored by applying at least one pigment.

Pigments within the meaning of the present disclosure are coloring compounds which have a solubility in water at 25° C. of less than 0.5 g/L, preferably less than 0.1 g/L, even more preferably less than 0.05 g/L. Water solubility can be determined, for example, by the method described below: 0.5 g of the pigment are weighed in a beaker. A stir-fish is added. Then one liter of distilled water is added. This mixture is heated to 25° C. for one hour while stirring on a magnetic stirrer. If undissolved components of the pigment are still visible in the mixture after this period, the solubility of the pigment is below 0.5 g/L. If the pigment-water mixture cannot be assessed visually due to the high intensity of the possibly finely dispersed pigment, the mixture is filtered. If a proportion of undissolved pigments remains on the filter paper, the solubility of the pigment is below 0.5 g/L.

Suitable color pigments can be of inorganic and/or organic origin. In a preferred embodiment, a process as contemplated herein is wherein the decolorizing agent is applied to keratin material that has been colored by application of at least one inorganic and/or organic pigment.

Preferred color pigments are selected from synthetic or natural inorganic pigments. Inorganic color pigments of natural origin can be produced, for example, from chalk, ochre, umber, green earth, burnt Terra di Siena or graphite. Furthermore, black pigments such as iron oxide black, colored pigments such as ultramarine or iron oxide red as well as fluorescent or phosphorescent pigments can be used as inorganic color pigments.

Particularly suitable are colored metal oxides, hydroxides and oxide hydrates, mixed-phase pigments, sulfur-comprising silicates, silicates, metal sulphides, complex metal cyanides, metal sulphates, chromates and/or molybdates. In particular, preferred color pigments are black iron oxide (CI 77499), yellow iron oxide (CI 77492), red and brown iron oxide (CI 77491), manganese violet (CI 77742), ultramarine (sodium aluminum sulfo silicates, CI 77007, pigment blue 29), chromium oxide hydrate (CI77289), iron blue (ferric ferrocyanides, CI77510) and/or carmine (cochineal).

As contemplated herein, colored pearlescent pigments are also particularly preferred color pigments. These are usually mica- and/or mica-based and can be coated with one or more metal oxides. Mica belongs to the layer silicates. The most important representatives of these silicates are muscovite, phlogopite, paragonite, biotite, lepidolite and margarite. To produce the pearlescent pigments in combination with metal oxides, the mica, mainly muscovite or phlogopite, is coated with a metal oxide.

As an alternative to natural mica, synthetic mica coated with one or more metal oxides can also be used as pearlescent pigment. Especially preferred pearlescent pigments are based on natural or synthetic mica (mica) and are coated with one or more of the metal oxides mentioned above. The color of the respective pigments can be varied by varying the layer thickness of the metal oxide(s).

In a preferred embodiment, a process as contemplated herein is wherein the decolorizing agent is applied to keratin material which has been colored by application of at least one inorganic pigment, the inorganic pigment preferably being selected from the group of colored metal oxides, metal hydroxides, metal oxide hydrates, silicates, metal sulfides, complex metal cyanides, metal sulfates, bronze pigments and/or mica- or mica-based colored pigments coated with at least one metal oxide and/or a metal oxychloride.

In a preferred embodiment, a process as contemplated herein is wherein the decolorizing agent is applied to keratin material which has been colored by the application of at least one pigment selected from mica- or mica-based pigments which have been colored with one or more metal oxides selected from the group of titanium dioxide (CI 77891), black iron oxide (CI 77499), yellow iron oxide (CI 77492), red and/or brown iron oxide (CI 77491, CI 77499), manganese violet (CI 77742), ultramarine (sodium aluminum sulfosilicates, CI 77007, Pigment Blue 29), chromium oxide hydrate (CI 77289), chromium oxide (CI 77288) and/or iron blue (ferric ferrocyanide, CI 77510)

Examples of particularly suitable color pigments are commercially available under the trade names Rona®, Colorona®, Xirona®, Dichrona® and Timiron® from Merck, Ariabel® and Unipure® from Sensient, Prestige® from Eckart Cosmetic Colors and Sunshine® from Sunstar.

Particularly preferred color pigments with the trade name Colorona® are, for example:

### Colorona Copper, Merck, MICA, CI 77491 (IRON OXIDES)

### Colorona Passion Orange, Merck, Mica, CI 77491 (Iron Oxides), Alumina

### Colorona Patina Silver, Merck, MICA, CI 77499 (IRON OXIDES), CI 77891 (TITANIUM DIOXIDE)

### Colorona RY, Merck, CI 77891 (TITANIUM DIOXIDE), MICA, CI 75470 (CARMINE)

### Colorona Oriental Beige, Merck, MICA, CI 77891 (TITANIUM DIOXIDE), CI 77491 (IRON OXIDES)

### Colorona Dark Blue, Merck, MICA, TITANIUM DIOXIDE, FERRIC FERROCYANIDE

### Colorona Chameleon, Merck, CI 77491 (IRON OXIDES), MICA

### Colorona Aborigine Amber, Merck, MICA, CI 77499 (IRON OXIDES), CI 77891 (TITANIUM DIOXIDE)

### Colorona Blackstar Blue, Merck, CI 77499 (IRON OXIDES), MICA

### Colorona Patagonian Purple, Merck, MICA, CI 77491 (IRON OXIDES), CI 77891 (TITANIUM DIOXIDE), CI 77510 (FERRIC FERROCYANIDE)

### Colorona Red Brown, Merck, MICA, CI 77491 (IRON OXIDES), CI 77891 (TITANIUM DIOXIDE)

### Colorona Russet, Merck, CI 77491 (TITANIUM DIOXIDE), MICA, CI 77891 (IRON OXIDES)

### Colorona Imperial Red, Merck, MICA, TITANIUM DIOXIDE (CI 77891), D&C RED NO. 30 (CI 73360)

### Colorona Majestic Green, Merck, CI 77891 (TITANIUM DIOXIDE), MICA, CI 77288 (CHROMIUM OXIDE GREENS)

### Colorona Light Blue, Merck, MICA, TITANIUM DIOXIDE (CI 77891), FERRIC FERROCYANIDE (CI 77510)

### Colorona Red Gold, Merck, MICA, CI 77891 (TITANIUM DIOXIDE), CI 77491 (IRON OXIDES)

### Colorona Gold Plus MP 25, Merck, MICA, TITANIUM DIOXIDE (CI 77891), IRON OXIDES (CI 77491)

### Colorona Carmine Red, Merck, MICA, TITANIUM DIOXIDE, CARMINE

### Colorona Blackstar Green, Merck, MICA, CI 77499 (IRON OXIDES)

### Colorona Bordeaux, Merck, MICA, CI 77491 (IRON OXIDES)

### Colorona Bronze, Merck, MICA, CI 77491 (IRON OXIDES)

### Colorona Bronze Fine, Merck, MICA, CI 77491 (IRON OXIDES)

### Colorona Fine Gold MP 20, Merck, MICA, CI 77891 (TITANIUM DIOXIDE), CI 77491 (IRON OXIDES)

### Colorona Sienna Fine, Merck, CI 77491 (IRON OXIDES), MICA

### Colorona Sienna, Merck, MICA, CI 77491 (IRON OXIDES)

Colorona Precious Gold, Merck, Mica, CI 77891 (Titanium dioxide), Silica, CI 77491 (Iron oxides), Tin oxide

### Colorona Sun Gold Sparkle MP 29, Merck, MICA, TITANIUM DIOXIDE, IRON OXIDES, MICA, CI 77891, CI 77491 (EU)

Colorona Mica Black, Merck, CI 77499 (Iron oxides), Mica, CI 77891 (Titanium dioxide)
Colorona Bright Gold, Merck, Mica, CI 77891 (Titanium dioxide), CI 77491 (Iron oxides)

### Colorona Blackstar Gold, Merck, MICA, CI 77499 (IRON OXIDES)

Other particularly preferred color pigments with the trade name Xirona® are for example:

### Xirona Golden Sky, Merck, Silica, CI 77891 (Titanium Dioxide), Tin Oxide

### Xirona Caribbean Blue, Merck, Mica, CI 77891 (Titanium Dioxide), Silica, Tin Oxide

### Xirona Kiwi Rose, Merck, Silica, CI 77891 (Titanium Dioxide), Tin Oxide

### Xirona Magic Mauve, Merck, Silica, CI 77891 (Titanium Dioxide), Tin Oxide.

In addition, particularly preferred color pigments with the trade name Unipure® are for example:

### Unipure Red LC 381 EM, Sensient CI 77491 (Iron Oxides), Silica

### Unipure Black LC 989 EM, Sensient, CI 77499 (Iron Oxides), Silica

### Unipure Yellow LC 182 EM, Sensient, CI 77492 (Iron Oxides), Silica

In another embodiment, the previously applied colorant may also contain one or more organic pigments.

The organic pigments as contemplated herein are correspondingly insoluble, organic dyes or color lacquers, which may be selected, for example, from the group of nitroso, nitro-azo, xanthene, anthraquinone, isoindolinone, isoindolinone, quinacridone, perinone, perylene, diketo-pyrrolopyorrole, indigo, thioindido, dioxazine and/or triarylmethane compounds.

Examples of particularly suitable organic pigments are carmine, quinacridone, phthalocyanine, sorghum, blue pigments with the Color Index numbers Cl 42090, CI 69800, CI 69825, CI 73000, CI 74100, CI 74160, yellow pigments with the Color Index numbers CI 11680, CI 11710, CI 15985, CI 19140, CI 20040, CI 21100, CI 21108, CI 47000, CI 47005, green pigments with the Color Index numbers CI 61565, CI 61570, CI 74260, orange pigments with the Color Index numbers CI 11725, CI 15510, CI 45370, CI 71105, red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

In a further particularly preferred embodiment, a process as contemplated herein, wherein the decolorizing agent is applied to keratin material which has been colored by application of at least one organic pigment, the organic pigment preferably being selected from the group of carmine, quinacridone, phthalocyanine, sorghum, blue pigments having the color index numbers Cl 42090, CI 69800, CI 69825, CI 73000, CI 74100, CI 74160, yellow pigments having the color index numbers CI 11680, CI 11710, CI 15985, CI 19140, CI 20040, CI 21100, CI 21108, CI 47000, CI 47005, green pigments with Color Index numbers CI 61565, CI 61570, CI 74260, orange pigments with Color Index numbers CI 11725, CI 15510, CI 45370, CI 71105, red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

The organic pigment can also be a color paint. As contemplated herein, the term color lacquer means particles comprising a layer of absorbed dyes, the unit of particle and dye being insoluble under the above mentioned conditions. The particles can, for example, be inorganic substrates, which can be aluminum, silica, calcium borosilate, calcium aluminum borosilicate or even aluminum.

For example, alizarin color varnish can be used.

Due to their excellent light and temperature resistance, the use of the above pigments in the agent is particularly preferred. It is also preferred if the pigments used have a certain particle size. As contemplated herein, it is therefore advantageous if the at least one pigment has an average particle size D₅₀ of 1.0 to 50 μm, preferably 5.0 to 45 μm, preferably 10 to 40 μm, in particular 14 to 30 μm. The mean particle size D₅₀, for example, can be determined using dynamic light scattering (DLS).

In the agent used for the preceding coloration in the process as contemplated herein, one or more pigments may be present, for example, in a total amount of from 0.01 to 10.0 wt. %, preferably from 0.1 to 5.0 wt. %, more preferably from 0.2 to 2.5 wt. % and very particularly preferably from 0.25 to 1.5 wt. %. Here, the quantities are based on the total quantity of all pigments used, which is set in relation to the total weight of the colorant.

In another very particularly preferred embodiment, a colorant as contemplated herein is wherein the colorant comprises—based on the total weight of the colorant—one or more pigments in a total amount of from 0.01 to 10.0 wt. %, preferably from 0.1 to 5.0 wt. %, more preferably from 0.2 to 2.5 wt. % and very particularly preferably from 0.25 to 1.5 wt. %.

As a further optional component, the colorants could also additionally contain one or more direct dyes. Direct-acting dyes are dyes that draw directly onto the hair and do not require an oxidative process to form the color. Direct dyes are usually nitrophenylene diamines, nitroaminophenols, azo dyes, anthraquinones, triarylmethane dyes or indophenols.

The direct dyes within the meaning of the present disclosure have a solubility in water (760 mmHg) at 25° C. of more than 0.5 g/L and are therefore not to be regarded as pigments.

Preferably, the direct dyes within the meaning of the present disclosure have a solubility in water (760 mmHg) at 25° C. of more than 1.0 g/L.

Direct dyes can be divided into anionic, cationic and non-ionic direct dyes.

In a further embodiment, an agent as contemplated herein may be wherein it additionally comprises at least one colorant compound selected from the group of anionic, nonionic and cationic direct dyes.

Suitable cationic direct dyes include Basic Blue 7, Basic Blue 26, HC Blue 16, Basic Violet 2 and Basic Violet 14, Basic Yellow 57, Basic Red 76, Basic Blue 16, Basic Blue 347 (Cationic Blue 347/Dystar), HC Blue No. 16, Basic Blue 99, Basic Brown 16, Basic Brown 17, Basic Yellow 57, Basic Yellow 87, Basic Orange 31, Basic Red 51 Basic Red 76.

As non-ionic direct dyes, non-ionic nitro and quinone dyes and neutral azo dyes can be used. Suitable non-ionic direct dyes are those listed under the international designations or Trade names HC Yellow 2, HC Yellow 4, HC Yellow 5, HC Yellow 6, HC Yellow 12, HC Orange 1, Disperse Orange 3, HC Red 1, HC Red 3, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9 known compounds, as well as 1,4-diamino-2-nitrobenzene, 2-amino-4-nitrophenol, 1,4-bis-(2-hydroxyethyl)-amino-2-nitrobenzene, 3-nitro-4-(2-hydroxyethyl)-aminophenol 2-(2-hydroxyethyl)amino-4,6-dinitrophenol, 4-[(2-hydroxyethyl)amino]-3-nitro-1-methylbenzene, 1-amino-4-(2-hydroxyethyl)-amino-5-chloro-2-nitrobenzene, 4-amino-3-nitrophenol, 1-(2′-ureidoethyl)amino-4-nitrobenzene, 2-[(4-amino-2-nitrophenyl)amino]benzoic acid, 6-nitro-1,2,3,4-tetrahydroquinoxaline, 2-hydroxy-1,4-naphthoquinone, picramic acid and its salts, 2-amino-6-chloro-4-nitrophenol, 4-ethylamino-3-nitrobenzoic acid and 2-chloro-6-ethylamino-4-nitrophenol.

Anionic direct dyes are also called acid dyes. Acid dyes are direct dyes that have at least one carboxylic acid group (—COOH) and/or one sulphonic acid group (—SO₃H). Depending on the pH value, the protonated forms (—COOH, —SO₃H) of the carboxylic acid or sulphonic acid groups are in equilibrium with their deprotonated forms (—COO⁻, —SO₃⁻ present). The proportion of protonated forms increases with decreasing pH. If direct dyes are used in the form of their salts, the carboxylic acid groups or sulphonic acid groups are present in deprotonated form and are neutralized with corresponding stoichiometric equivalents of cations to maintain electro neutrality. Inventive acid dyes can also be used in the form of their sodium salts and/or their potassium salts.

The acid dyes within the meaning of the present disclosure have a solubility in water (760 mmHg) at 25° C. of more than 0.5 g/L and are therefore not to be regarded as pigments. Preferably the acid dyes within the meaning of the present disclosure have a solubility in water (760 mmHg) at 25° C. of more than 1.0 g/L.

The alkaline earth salts (such as calcium salts and magnesium salts) or aluminum salts of acid dyes often have a lower solubility than the corresponding alkali salts. If the solubility of these salts is below 0.5 g/L (25° C., 760 mmHg), they do not fall under the definition of a direct dye.

An essential characteristic of acid dyes is their ability to form anionic charges, whereby the carboxylic acid or sulphonic acid groups responsible for this are usually linked to different chromophoric systems. Suitable chromophoric systems can be found, for example, in the structures of nitrophenylenediamines, nitroaminophenols, azo dyes, anthraquinone dyes, triarylmethane dyes, xanthene dyes, rhodamine dyes, oxazine dyes and/or indophenol dyes.

In a further embodiment, an agent for dyeing keratinous material may be wherein it comprises at least one anionic direct dye selected from the group of the nitrophenylenediamines, the nitroaminophenols, the azo dyes, the anthraquinone dyes, the triarylmethane dyes, the xanthene dyes the rhodamine dyes, the oxazine dyes and/or the indophenol dyes, the dyes from the abovementioned group each having at least one carboxylic acid group (—COOH), a sodium carboxylate group (—COONa), a potassium carboxylate group (—COOK), a sulfonic acid group (—SO₃H), a sodium sulfonate group (—SO₃Na) and/or a potassium sulfonate group (—SO₃K).

Suitable acid dyes may include, for example, one or more compounds selected from the following group: Acid Yellow 1 (D&C Yellow 7, Citronin A, Ext. D&C Yellow No. 7, Japan Yellow 403, CI 10316, COLIPA no B001), Acid Yellow 3 (COLIPA no: C 54, D&C Yellow No 10, Quinoline Yellow, E104, Food Yellow 13), Acid Yellow 9 (CI 13015), Acid Yellow 17 (CI 18965), Acid Yellow 23 (COLIPA no C 29, Covacap Jaune W 1100 (LCW), Sicovit Tartrazine 85 E 102 (BASF), Tartrazine, Food Yellow 4, Japan Yellow 4, FD&C Yellow No. 5), Acid Yellow 36 (CI 13065), Acid Yellow 121 (CI 18690), Acid Orange 6 (CI 14270), Acid Orange 7 (2-Naphthol orange, Orange II, CI 15510, D&C Orange 4, COLIPA no C015), Acid Orange 10 (C.I. 16230; Orange G sodium salt), Acid Orange 11 (CI 45370), Acid Orange 15 (CI 50120), Acid Orange 20 (CI 14600), Acid Orange 24 (BROWN 1; CI 20170; KATSU201; nosodiumsalt; Brown No. 201; RESORCIN BROWN; ACID ORANGE 24; Japan Brown 201; D & C Brown No. 1), Acid Red 14 (C.I.14720), Acid Red 18 (E124, Red 18; CI 16255), Acid Red 27 (E 123, CI 16185, C-Rot 46, Real red D, FD&C Red Nr.2, Food Red 9, Naphthol red S), Acid Red 33 (Red 33, Fuchsia Red, D&C Red 33, CI 17200), Acid Red 35 (CI C.I.18065), Acid Red 51 (CI 45430, Pyrosin B, Tetraiodfluorescein, Eosin J, Iodeosin), Acid Red 52 (CI 45100, Food Red 106, Solar Rhodamine B, Acid Rhodamine B, Red no 106 Pontacyl Brilliant Pink), Acid Red 73 (CI 27290), Acid Red 87 (Eosin, CI 45380), Acid Red 92 (COLIPA no C53, CI 45410), Acid Red 95 (CI 45425, Erythtosine, Simacid Erythrosine Y), Acid Red 184 (CI 15685), Acid Red 195, Acid Violet 43 (Jarocol Violet 43, Ext. D&C Violet no 2, C.I. 60730, COLIPA no C063), Acid Violet 49 (CI 42640), Acid Violet 50 (CI 50325), Acid Blue 1 (Patent Blue, CI 42045), Acid Blue 3 (Patent Blue V, CI 42051), Acid Blue 7 (CI 42080), Acid Blue 104 (CI 42735), Acid Blue 9 (E 133, Patent Blue AE, Amido blue AE, Erioglaucin A, CI 42090, C.I. Food Blue 2), Acid Blue 62 (CI 62045), Acid Blue 74 (E 132, CI 73015), Acid Blue 80 (CI 61585), Acid Green 3 (CI 42085, Foodgreen1), Acid Green 5 (CI 42095), Acid Green 9 (C.I.42100), Acid Green 22 (C.I.42170), Acid Green 25 (CI 61570, Japan Green 201, D&C Green No. 5), Acid Green 50 (Brilliant Acid Green BS, C.I. 44090, Acid Brilliant Green BS, E 142), Acid Black 1 (Black no 401, Naphthalene Black 10B, Amido Black 10B, CI 20 470, COLIPA no B15), Acid Black 52 (CI 15711), Food Yellow 8 (CI 14270), Food Blue 5, D&C Yellow 8, D&C Green 5, D&C Orange 10, D&C Orange 11, D&C Red 21, D&C Red 27, D&C Red 33, D&C Violet 2 and/or D&C Brown 1.

For example, the water solubility of anionic direct dyes can be determined in the following way. 0.1 g of the anionic direct dye is placed in a beaker. A stir-fish is added. Then add 100 ml of water. This mixture is heated to 25° C. on a magnetic stirrer while stirring. It is stirred for 60 minutes. The aqueous mixture is then visually assessed. If there are still undissolved radicals, the amount of water is increased—for example in steps of 10 ml. Water is added until the amount of dye used is completely dissolved. If the dye-water mixture cannot be assessed visually due to the high intensity of the dye, the mixture is filtered. If a proportion of undissolved dyes remains on the filter paper, the solubility test is repeated with a higher quantity of water. If 0.1 g of the anionic direct dye dissolves in 100 ml water at 25° C., the solubility of the dye is 1.0 g/L.

Acid Yellow 1 is called 8-hydroxy-5,7-dinitro-2-naphthalenesulfonic acid disodium salt and has a solubility in water of at least 40 g/L (25° C.).
Acid Yellow 3 is a mixture of the sodium salts of mono- and sisulfonic acids of 2-(2-quinolyl)-1H-indene-1,3(2H)-dione and has a water solubility of 20 g/L (25° C.).
Acid Yellow 9 is the disodium salt of 8-hydroxy-5,7-dinitro-2-naphthalenesulfonic acid, its solubility in water is above 40 g/L (25° C.).
Acid Yellow 23 is the trisodium salt of 4,5-dihydro-5-oxo-1-(4-sulfophenyl)-4-((4-sulfophenyl)azo)-1H-pyrazole-3-carboxylic acid and is highly soluble in water at 25° C.
Acid Orange 7 is the sodium salt of 4-[(2-hydroxy-1-naphthyl)azo]benzene sulphonate. Its water solubility is more than 7 g/L (25° C.).
Acid Red 18 is the trinatirum salt of 7-hydroxy-8-[(E)-(4-sulfonato-1-naphthyl)-diazenyl)]-1,3-naphthalene disulfonate and has a very high water solubility of more than 20 wt. %.
Acid Red 33 is the diantrium salt of 5-amino-4-hydroxy-3-(phenylazo)-naphthalene-2,7-disulphonate, its solubility in water is 2.5 g/L (25° C.).
Acid Red 92 is the disodium salt of 3,4,5,6-tetrachloro-2-(1,4,5,8-tetrabromo-6-hydroxy-3-oxoxanthen-9-yl)benzoic acid, whose solubility in water is indicated as greater than 10 g/L (25° C.).
Acid Blue 9 is the disodium salt of 2-({4-[N-ethyl(3-sulfonatobenzyl]amino]phenyl} {4-[(N-ethyl(3-sulfonatobenzyl)imino]-2,5-cyclohexadien-1-ylidene}methyl)-benzenesulfonate and has a solubility in water of more than 20 wt. % (25° C.).

In a further embodiment, a colorant as contemplated herein is therefore wherein it comprises at least one direct dye selected from the group of Acid Yellow 1, Acid Yellow 3, Acid Yellow 9, Acid Yellow 17, Acid Yellow 23, Acid Yellow 36, Acid Yellow 121, Acid Orange 6, Acid Orange 7, Acid Orange 10, Acid Orange 11, Acid Orange 15, Acid Orange 20, Acid Orange 24, Acid Red 14, Acid Red 27, Acid Red 33, Acid Red 35, Acid Red 51, Acid Red 52, Acid Red 73, Acid Red 87, Acid Red 92, Acid Red 95, Acid Red 184, Acid Red 195, Acid Violet 43, Acid Violet 49, Acid Violet 50, Acid Blue 1, Acid Blue 3, Acid Blue 7, Acid Blue 104, Acid Blue 9, Acid Blue 62, Acid Blue 74, Acid Blue 80, Acid Green 3, Acid Green 5, Acid Green 9, Acid Green 22, Acid Green 25, Acid Green 50, Acid Black 1, Acid Black 52, Food Yellow 8, Food Blue 5, D&C Yellow 8, D&C Green 5, D&C Orange 10, D&C Orange 11, D&C Red 21, D&C Red 27, D&C Red 33, D&C Violet 2 and/or D&C Brown 1.

The direct-acting dye or dyes can be used in various amounts in the colorant, depending on the desired color intensity. Good results could be obtained if the colorant—based on the total weight of the colorant—comprises one or more direct dyes in a total amount of 0.01 to 10.0 wt. %, preferably 0.1 to 8.0 wt. %, more preferably 0.2 to 6.0 wt. % and most preferably 0.5 to 4.5 wt. %.

Furthermore, the agent may also contain, as an additional optional component, a coloring compound selected from the group of photochromic or thermochromic dyes.

Photochromic dyes are dyes that react to irradiation with UV light (sunlight or black light) with a reversible change in hue. In this process, the UV light changes the chemical structure of the dyes and thus their absorption behavior (photochromism).

Thermochromic dyes are dyes that react to temperature changes with a reversible change in hue. In this process, the change in temperature alters the chemical structure of the dyes and thus their absorption behavior (Thermochromism).

The colorant may contain—based on the total weight of the colorant—one or more photochromic and/or thermochromic dyes in a total amount of from 0.01 to 10.0 wt. %, preferably from 0.1 to 8.0 wt. %, more preferably from 0.2 to 6.0 wt. % and most preferably from 0.5 to 4.5 wt. %.

### Decolorizing Agent

In the process as contemplated herein, a decolorizing agent is applied to the keratin material, in particular to human hair, which has been dyed as described above. The decolorizing agent is applied to the dyed keratin material and rinsed off again after a reaction time.

The time when the decolorizer is applied depends on the needs of the user and can be adjusted to his habits.

For example, it is possible to apply the decolorizing agent to the freshly dyed, still wet or preferably dried keratin material, so that a period of several hours lies between the rinsing out of the dyeing agent and the application of the decolorizing agent. This application of the decolorizing agent shortly after dyeing can be done especially if the result of the dyeing does not meet the user's expectations. It is also possible, for example, to apply a very intense or eye-catching coloration for a particular occasion and then remove the coloration after that occasion.

Likewise, it is possible that there is a longer period of time between the previous application of the colorant and the application of the decolorant, which can range from a few to several days or even weeks. In this context, the requirement is that the decolorizing agent is applied to colored keratin material, which means that the keratin material must still be colored by the application of the pigments.

The decolorizing agent is wherein it comprises

(a) comprises at least one anionic surfactant, and

(b) has a pH value of 1.0 to 3.5.

### Anionic Surfactants (a) in the Decolorant

As an essential ingredient, the decolorant as contemplated herein comprises at least one anionic surfactant (a).

The term surfactants (T) refer to surface-active substances that can form adsorption layers on surfaces and interfaces or aggregate in bulk phases to form micelle colloids or lyotropic mesophases. A distinction is made between anionic surfactants comprising a hydrophobic radical and a negatively charged hydrophilic head group, amphoteric or zwitterionic surfactants, which carry both a negative and a compensating positive charge, cationic surfactants, which have a positively charged hydrophilic group in addition to a hydrophobic radical, and nonionic surfactants, which have no charges but strong dipole moments and are strongly hydrated in aqueous solution.

Suitable anionic surfactants (a) that can be used in the decolorizing agent as contemplated herein include:
linear and branched fatty acids with 8 to 30 C atoms (Soaps),
Ethercarboxylic acids of the formula R—O—(CH₂—CH₂O)ₓ—CH₂—COOH, in which R is a linear or branched, saturated or unsaturated alkyl group having 8 to 30 C atoms and x=0 or 1 to 16
Acylsarcosides with 8 to 24 C atoms in the acyl group,
Acyltaurides with 8 to 24 C atoms in the acyl group,
Acyl isethionates with 8 to 24 C atoms in the acyl group,
Sulfosuccinic acid mono- and/or dialkyl esters with 8 to 24 C atoms in the alkyl group and sulfosuccinic acid mono-alkyl polyoxyethyl esters with 8 to 24 C atoms in the alkyl group and 1 to 6 oxyethyl groups,
Alpha-olefin sulfonates with 8 to 24 C atoms,
Alkyl sulfate and/or alkyl polyglycol ether sulfate salts of the formula R—(OCH₂—CH₂)ₓ—OSO₃⁻X⁺, in which R is a preferably linear or branched, saturated or unsaturated alkyl group having 8 to 30 carbon atoms, x=0 or 1 to 12 and X is an alkali metal or ammonium ion,
Sulfonates of unsaturated fatty acids with 8 to 24 C atoms and 1 to 6 double bonds,
Esters of tartaric acid and citric acid with alcohols, which are addition products of about 2-15 molecules of ethylene oxide and/or propylene oxide to fatty alcohols with 8 to 22 C atoms,
Alkyl and/or alkenyl ether phosphates of the formula,

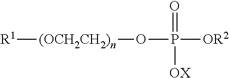

where R¹ is preferably an aliphatic hydrocarbon radical of 8 to 30 carbon atoms, R² is hydrogen, a radical (CH₂CH₂O)ₙR¹ or X, n is from 0 to 10 and X is hydrogen, an alkali metal or alkaline earth metal or NR³R⁴R⁵R⁶, where R³ to R⁶ are each independently of the others a C₁- to C₄-hydrocarbon radical.

Particularly good results were obtained when at least one anionic surfactant (a) of formula (T-1) was used in the decolorant,

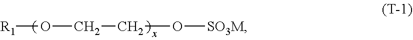

where

R₁ represents a linear or branched, saturated or unsaturated C₈-C₃₀ alkyl group,
x stands for an integer from 0 to 50, and
M is a hydrogen atom, ammonium (NH₄)₊ or an equivalent of a monovalent or polyvalent cation.

In a very particularly preferred embodiment, a process as contemplated herein is A process wherein the decolorizing agent comprises at least one anionic surfactant (a) of the formula (T-1),

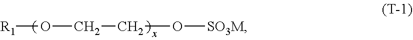

where

R₁ represents a linear or branched, saturated or unsaturated C₈-C₃₀ alkyl group,
x stands for an integer from 0 to 50, and
M is a hydrogen atom, ammonium (NH₄)₊ or an equivalent of a monovalent or polyvalent cation.

The radical R₁ represents the hydrophobic part of the anionic surfactant and stands for a linear or branched, saturated or unsaturated C₈-C₃₀ alkyl group. If the radical stands for an unsaturated C₈-C₃₀ alkyl group, then the alkyl group can be mono- or polyunsaturated.

Preferably, the radical R1 is a linear, saturated or unsaturated C₈-C₃₀ alkyl group-Very preferably, the radical R1 is a linear, saturated or unsaturated C₁₂-C₂₂ alkyl group. Explicitly quite particularly preferred, the radical R1 stands for an R for a linear, saturated or unsaturated C₁₂-C₁₈ alkyl group.

Examples of saturated, linear C₈-C₃₀ alkyl groups are the lauryl group, the myristyl group, the cetyl group, the stearyl group, and the behenyl group.

The index number x indicates the number of ethylene oxide groups included in the anionic surfactant. If x is 0, the anionic surfactant of formula (T-1) has no ethylene oxide units, in which case an alkyl sulfate or the salt of an alkyl sulfate is present.

Examples of the salts of alkyl sulfates are sodium lauryl sulfate and sodium myristyl sulfate.

Preferably, x represents an integer from 0 to 5, and particularly preferably, x represents an integer from 1 to 5.

The radical M stands for a hydrogen atom, for ammonium (NH₄)₊ or for an equivalent of a monovalent or polyvalent cation.

If M stands for a hydrogen atom, then the anionic surfactant is present in the form of the protonated (and possibly ethoxylated) sulfuric acid ester. In aqueous solution, the protonated form is in equilibrium with the deprotonated form, with the deprotonated form carrying an anionic charge. For this reason, the protonated compounds of formula (I) also fall into the group of anionic surfactants.

If M stands for ammonium (NH₄)₊ or for an equivalent of a monovalent or polyvalent cation, then the anionic surfactant of formula (I) is present in the form of its salt. The presence of the corresponding equivalent of a monovalent or polyvalent cation here ensures the electroneutrality of the anion surfactant. Preferably, M represents a monovalent cation, in particular a sodium or potassium cation.

In a very particularly preferred embodiment, a process as contemplated herein is The process wherein the decolorant comprises at least one anionic surfactant (a) of the formula (T-1) where

R1 is a linear, saturated or unsaturated C₁₂-C₁₈ alkyl group,
X is an integer from 0 to 5, preferably an integer from 1 to 5,
M represents a hydrogen atom or an alkali metal cation, preferably a sodium cation or a potassium cation.

A particularly preferred anionic surfactant of formula (I) can be obtained commercially, for example, under the trade name Texapon NSO BZ (BZ=preserved with benzoic acid) and the INCI designation Sodium Laureth Sulfate from BASF. This Sodium Laureth Sulfate has the CAS number 68891-38-3.

Furthermore, a decolorizing agent with good decolorizing performance could be obtained when the decolorizing agent as contemplated herein included at least one anionic surfactant (a) of formula (T-2),

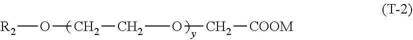

where
R² represents a linear or branched, saturated or unsaturated C₈-C₃₀ alkyl group,
y stands for an integer from 0 to 16,
M is hydrogen or a metal such as an alkali metal, in particular sodium, potassium, lithium, an alkaline earth metal, in particular 12 magnesium, 12 calcium, 12 zinc, or an ammonium ion (NH4⁺).

In another preferred embodiment, a process as contemplated herein is wherein the decolorizing agent comprises at least one anionic surfactant (a) of the formula (T-2),

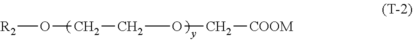

where
R² represents a linear or branched, saturated or unsaturated C₈-C₃₀ alkyl group,
y stands for an integer from 0 to 16,
M is hydrogen or a metal such as an alkali metal, in particular sodium, potassium, lithium, an alkaline earth metal, in particular 12 magnesium, 12 calcium, 12 zinc, or an ammonium ion (NH₄+).

The radical R² represents the hydrophobic part of the anionic surfactant of the formula (T-2) and stands for a linear or branched, saturated or unsaturated C₈-C₃₀ alkyl group. If the radical stands for an unsaturated C₈-C₃₀ alkyl group, then the alkyl group can be mono- or polyunsaturated.

Preferably, the radical R² is a linear, saturated or unsaturated C₈-C₃₀ alkyl group-Very preferably, the radical R² is a linear, saturated or unsaturated C₁₂-C₂₂ alkyl group. Explicitly quite particularly preferred, the radical R² stands for an R for a linear, saturated or unsaturated C₁₂-C₁₈ alkyl group.

Examples of saturated, linear C₈-C₃₀ alkyl groups are the lauryl group, the myristyl group, the cetyl group, the stearyl group, and the behenyl group.

The Index number y indicates the number of ethylenoyide groups included in the anionic surfactant. If y is 0, then the anionic surfactant of formula (T-2) has no ethylene oxide units, in which case an alkyl sulfate or the salt of an alkyl sulfate is present.

Preferably, y represents an integer from 0 to 6, and particularly preferably, y represents an integer from 1 to 6.

The radical M stands for a hydrogen atom, for ammonium (NH₄)₊ or for an equivalent of a monovalent or polyvalent cation.

If M stands for a hydrogen atom, then the anionic surfactant is present in the form of the protonated (and possibly ethoxylated) sulfuric acid ester. In aqueous solution, the protonated form is in equilibrium with the deprotonated form, with the deprotonated form carrying an anionic charge. For this reason, the protonated compounds of formula (I) also fall into the group of anionic surfactants.

If M stands for ammonium (NH₄)₊ or for an equivalent of a monovalent or polyvalent cation, then the anionic surfactant of formula (I) is present in the form of its salt. The presence of the corresponding equivalent of a monovalent or polyvalent cation here ensures the electroneutrality of the anion surfactant. Preferably, M represents a monovalent cation, in particular a sodium or potassium cation.

An example of a particularly well-suited anionic surfactant of formula (T-2) is sodium laureth-6 carboxylate, which can be obtained commercially under the trade name Akypo Soft 45 HP from the Kao company. The surfactant is also known as alkyl ether carboxylic acid sodium salt and has the CAS number 33939-64-9.

To ensure particularly good color results in the process as contemplated herein, the decolorizing agent comprises the anionic surfactant(s) (a) preferably in certain quantity ranges. Particularly intense and uniform colorations could be obtained if the decolorant—based on the total weight of the decolorant—included one or more anionic surfactants (a) in a total amount of 1.0 to 15.0 wt. %, preferably 1.5 to 13.0 wt. %, more preferably 3.0 to 12.0 wt. % and very particularly preferably 6.0 to 11.0 wt. %.

In a very particularly preferred embodiment, a process as contemplated herein is A process wherein the decolorant comprises—based on the total weight of the decolorant—one or more anionic surfactants (a) in a total amount of 1.0 to 15.0 wt. %, preferably 1.5 to 13.0 wt. %, more preferably 3.0 to 12.0 wt. % and very particularly preferably 6.0 to 11.0 wt. %.

### Water Content in Decolorizing Agent

Since the decolorant is adjusted to an acidic pH in the range of 1.0 to 3.5 as contemplated herein, it comprises water or comprises a water-comprising carrier.

Accordingly, a first object of the present disclosure is, in other words, a process for decolorizing keratin material which has been colored by application of at least one pigment, wherein a decolorizing agent which is

(a) comprises at least one anionic surfactant, and

(b) comprises water and has a pH value of 1.0 to 3.5,

is applied to the dyed keratin material and rinsed off again after a contact time.

Particularly good removal of the excess pigments or amino silicones was possible if the decolorant—based on the total weight of the decolorant—had a water content of 50 to 99 wt. %, preferably 55 to 98 wt. %, more preferably 60 to 97 wt. %, and particularly preferably 70 to 96 wt. %.

In a further particularly preferred embodiment, a process as contemplated herein is therefore wherein the decolorant—based on the total weight of the decolorant—has a water content of from 50 to 99 wt. %, preferably from 55 to 98 wt. %, more preferably from 60 to 97 wt. %, and particularly preferably from 70 to 96 wt. %.

### pH Value of the Decolorant

The decolorant is exemplified by an acidic pH value in the range of 1.0 to 3.5.

In the series of tests leading to this present disclosure, it was shown that by selecting the optimum pH value, the decolorizing performance could be increased. A good decolorizing effect could already be observed from a pH value of 3.5. However, by further lowering the pH, this decolorizing performance could be further improved.

In this context, it has been found to be particularly preferred if the decolorant has a pH value (b) of 1.5 to 3.3, preferably 1.8 to 3.0 and especially preferably 2.0 to 3.0.

In the context of a further very particularly preferred embodiment, a process as contemplated herein is wherein the decolorizing agent has a pH (b) of from 1.5 to 3.3, preferably from 1.8 to 3.0 and particularly preferably from 2.0 to 3.0.

### Application of the Decolorizing Agent

Within the process as contemplated herein, the decolorizing agent is applied to the colored keratin material and rinsed off again after a reaction time.

Since the decolorizing agent is applied to the colored hair, the decolorizing agent must be applied to the keratin material after the application of the previously described colorant.

In other words, the decolorizing agent is applied to the keratin material after the colorant has been rinsed out and the keratin material has been dried, preferably for accurate determination of the color result.

The exact time of application of the decolorizing agent is determined by the user's wish to remove the unwanted or no longer required coloration. For example, the decolorizing agent can be applied to the dyed keratin material 12 to 24 hours after application of the dyeing agent. In a further embodiment, however, the user may wear the colored keratin materials, in particular the hair, for a period of several days to weeks until he decides to change the coloration again or wants his original hair color back.

### Other Surfactants in the Decolorant

In addition to the zwitterionic or amphoteric surfactants (a) described above, the decolorant as contemplated herein may optionally also contain further surfactants.

In another very particularly preferred embodiment, a process as contemplated herein is wherein the decolorizing agent comprises at least one cationic, nonionic and/or zwitterionic or amphoteric surfactant.

The term surfactants (T) refer to surface-active substances that can form adsorption layers on surfaces and interfaces or aggregate in bulk phases to form micelle colloids or lyotropic mesophases. A distinction is made between anionic surfactants comprising a hydrophobic radical and a negatively charged hydrophilic head group, amphoteric surfactants, which carry both a negative and a compensating positive charge, cationic surfactants, which in addition to a hydrophobic radical have a positively charged hydrophilic group, and non-ionic surfactants, which have no charges but strong dipole moments and are strongly hydrated in aqueous solution.

Cationic surfactants are surfactants, i.e., surface-active compounds, each with one or more positive charges. Cationic surfactants contain only positive charges. Usually, these surfactants are composed of a hydrophobic part and a hydrophilic head group, the hydrophobic part usually comprising a hydrocarbon backbone (e.g., comprising one or two linear or branched alkyl chains) and the positive charge(s) being located in the hydrophilic head group. Examples of cationic surfactants are
quaternary ammonium compounds which, as hydrophobic radicals, may carry one or two alkyl chains with a chain length of 8 to 28 C atoms,
quaternary phosphonium salts substituted with one or more alkyl chains with a chain length of 8 to 28 C atoms or
tertiary sulfonium salts.

Furthermore, the cationic charge can also be part of a heterocyclic ring (e.g., an imidazolium ring or a pyridinium ring) in the form of an onium structure. In addition to the functional unit carrying the cationic charge, the cationic surfactant may also contain other uncharged functional groups, as is the case for example with esterquats. The cationic surfactants are used in a total quantity of 0.1 to 45 wt. %, preferably 1 to 30 wt. % and most preferably 1 to 15 wt. %—based on the total weight of the respective agent.

Non-ionic surfactants contain, for example, a polyol group, a polyalkylene glycol ether group or a combination of polyol and polyglycol ether group as the hydrophilic group. Such links include
Addition products of 2 to 50 mol ethylene oxide and/or 0 to 5 mol propylene oxide to linear and branched fatty alcohols with 6 to 30 C atoms, the fatty alcohol polyglycol ethers or the fatty alcohol polypropylene glycol ethers or mixed fatty alcohol polyethers,
Addition products of 2 to 50 mol ethylene oxide and/or 0 to 5 mol propylene oxide to linear and branched fatty acids with 6 to 30 C atoms, the fatty acid polyglycol ethers or the fatty acid polypropylene glycol ethers or mixed fatty acid polyethers,
Addition products of 2 to 50 mol ethylene oxide and/or 0 to 5 mol propylene oxide to linear and branched alkylphenols having 8 to 15 C atoms in the alkyl group, the alkylphenol polyglycol ethers or the alkylpolypropylene glycol ethers or mixed alkylphenol polyethers,
with a methyl or C₂-C₆-alkyl radical end-group capped addition products of 2 to 50 moles of ethylene oxide and/or 0 to 5 moles of propylene oxide to linear and branched fatty alcohols with 8 to 30 C atoms, to fatty acids with 8 to 30 C atoms and to alkylphenols with 8 to 15 C atoms in the alkyl group, such as the grades available under the sales names Dehydol® LS, Dehydol® LT (Cognis),
C₁₂-C₃₀ fatty acid mono- and diesters of addition products of 1 to 30 mol ethylene oxide to glycerol,
Addition products of 5 to 60 mol ethylene oxide to castor oil and hardened castor oil,
Polyol fatty acid esters, such as the commercial product Hydagen® HSP (Cognis) or Sovermol® grades (Cognis),
alkoxylated triglycerides,
alkoxylated fatty acid alkyl esters of the formula (Tnio-1)

R¹CO—(OCH₂CHR²)_{w}OR³ (Tnio-1)

in which R¹CO is a linear or branched, saturated and/or unsaturated acyl radical having 6 to 22 carbon atoms, R² is hydrogen or methyl, R³ is linear or branched alkyl radicals having 1 to 4 carbon atoms and w is numbers from 1 to 20,
amine oxides,
Hydroxy mixed ethers, as described for example in DE-OS 19738866,
Sorbitan fatty acid esters and addition products of ethylene oxide to sorbitan fatty acid esters such as polysorbates,
Sugar fatty acid esters and addition products of ethylene oxide to sugar fatty acid ester,
Addition products of ethylene oxide to fatty acid alkanolamides and fatty amines,
Sugar tensides of the alkyl and alkenyl oligoglycoside type according to formula (E4-II),

R⁴O-[G]ₚ (Tnio-2)

in which R⁴ is an alkyl or alkenyl radical comprising 4 to 22 carbon atoms, G is a sugar residue comprising 5 or 6 carbon atoms and p is a number of 1 to 10. They can be obtained by the relevant methods of preparative organic chemistry. The alkyl and alkenyl oligoglycosides can be derived from aldoses or ketoses with 5 or 6 carbon atoms, preferably glucose. The preferred alkyl and/or alkenyl oligoglycosides are thus alkyl and/or alkenyl oligoglucosides. The index number p in the general formula (Tnio-2) indicates the degree of oligomerization (DP), i.e., the distribution of mono- and oligoglycosides and stands for a number between 1 and 10. While p must always be an integer in the individual molecule and can assume the values p=1 to 6, the value p for a certain alkyl oligoglycoside is an analytically determined arithmetical quantity, which usually represents a fractional number. Preferably alkyl and/or alkenyl oligoglycosides with an average degree of oligomerization p of 1.1 to 3.0 are used. From an application technology point of view, those alkyl and/or alkenyl oligoglycosides are preferred whose degree of oligomerization is less than 1.7 and in particular lies between 1.2 and 1.4. The alkyl or alkenyl radical R⁴ can be derived from primary alcohols comprising 4 to 11, preferably 8 to 10 carbon atoms. Typical examples are butanol, caproic alcohol, caprylic alcohol, caprin alcohol and undecrylic alcohol as well as their technical mixtures, such as those obtained in the hydrogenation of technical fatty acid methyl esters or in the course of the hydrogenation of aldehydes from Roelen's oxo synthesis. Preferred are alkyl oligoglucosides with a chain length of C₈-C₁₀ (DP=1 to 3), which are obtained as a preliminary step in the distillative separation of technical C₈-C₁₈ coconut-fatty alcohol and may be contaminated with less than 6 wt. % of C₁₂ alcohol, and alkyl oligoglucosides based on technical C_{9/11} oxoalcohols (DP=1 to 3). The alkyl or alkenyl radical R¹⁵ can also be derived from primary alcohols having 12 to 22, preferably 12 to 14 carbon atoms. Typical examples are lauryl alcohol, myristyl alcohol, cetyl alcohol, palmoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol, brassidyl alcohol and their technical mixtures, which can be obtained as described above. Preferred are alkyl oligoglucosides based on hardened C12/14 coconut alcohol with a DP of 1 to 3.
Sugar surfactants of the fatty acid N-alkyl polyhydroxyalkylamide type, a nonionic surfactant of formula (Tnio-3)

R⁵CO—NR⁶—[Z] (Tnio-3)

in which R⁵CO is an aliphatic acyl radical comprising 6 to 22 carbon atoms, R⁶ is hydrogen, an alkyl or hydroxyalkyl radical comprising 1 to 4 carbon atoms and [Z] is a linear or branched polyhydroxyalkyl radical comprising 3 to 12 carbon atoms and 3 to 10 hydroxyl groups. The fatty acid N-alkyl polyhydroxyalkylamides are known substances that can usually be obtained by reductive amination of a reducing sugar with ammonia, an alkylamine or an alkanolamine and subsequent acylation with a fatty acid, a fatty acid alkyl ester or a fatty acid chloride. The fatty acid N-alkyl polyhydroxyalkylamides are preferably derived from reducing sugars with 5 or 6 carbon atoms, especially from glucose. The preferred fatty acid N-alkyl polyhydroxyalkylamides are therefore fatty acid N-alkylglucamides as represented by the formula (Tnio-4):

R⁷CO—(NR⁸)—CH₂—[CH(OH)]₄—CH₂OH (Tnio-4)

Preferably, glucamides of the formula (Tnio-4) are used as fatty acid-N-alkyl polyhydroxyalkylamides, in which R⁸ represents hydrogen or an alkyl group and R⁷CO represents the acyl radical of caproic acid, caprylic acid, capric acid, Lauric acid, myristic acid, palmitic acid, palmoleic acid, stearic acid, isostearic acid, oleic acid, elaidic acid, petroselinic acid, linoleic acid, linolenic acid, arachidic acid, gadoleic acid, behenic acid or erucic acid or their technical mixtures. Particularly preferred are fatty acid N-alkyl glucamides of the formula (Tnio-4), which are obtained by reductive amination of glucose with methylamine and subsequent acylation with lauric acid or C12/14 coconut fatty acid or a corresponding derivative. Furthermore, polyhydroxyalkylamides can also be derived from maltose and palatinose.

Other typical examples of nonionic surfactants are fatty acid amide polyglycol ethers, fatty amine polyglycol ethers, mixed ethers or mixed formals, protein hydrolysates (especially wheat-based vegetable products) and polysorbates.

The alkylene oxide addition products to saturated linear fatty alcohols and fatty acids, each with 2 to 30 moles of ethylene oxide per mole of fatty alcohol or fatty acid, and the sugar surfactants have proved to be preferred nonionic surfactants. Preparations with excellent properties are also obtained if they contain fatty acid esters of ethoxylated glycerol as non-ionic surfactants.

Preferred zwitterionic surfactants include betaines, N-alkyl-N,N-dimethylammonium glycinate, N-acyl-aminopropyl-N,N-dimethylammonium glycinate, and 2-alkyl-3-carboxymethyl-3-hydroxyethyl-imidazolines. A preferred zwitterionic surfactant is known under the INCI name Cocamidopropyl betaine.

### Other Optional Ingredients in the Decolorant

In addition to the ingredients essential to the present disclosure already described, the decolorant may also contain other optional ingredients, such as solvents, anionic, nonionic, zwitterionic and/or cationic polymers; structurants such as glucose, maleic acid and lactic acid, hair-conditioning compounds such as phospholipids, for example lecitin and cephalins; perfume oils, dimethyl isosorbide and cyclodextrins; fiber structure-improving agents, in particular mono-, di- and oligosaccharides such as glucose, galactose, fructose, fructose and lactose; dyes for coloring the agent; antidandruff agents such as piroctone olamine, zinc omadine and climbazole; amino acids and oligopeptides; protein hydrolysates on animal and/or vegetable basis, as well as in the form of their fatty acid condensation products or, optionally, anionically or cationically modified derivatives; vegetable oils; light stabilizers and UV blockers; active ingredients such as panthenol, pantothenic acid, pantolactone, allantoin, pyrrolidinonecarboxylic acids and their salts, and bisabolol; Polyphenols, in particular hydroxycinnamic acids, 6,7-dihydroxycoumarins, hydroxybenzoic acids, catechins, tannins, leucoanthocyanidins, anthocyanidins, flavanones, flavones and flavonols; ceramides or pseudoceramides; vitamins, provitamins and vitamin precursors; plant extracts; Fats and waxes such as fatty alcohols, beeswax, montan wax and kerosene; swelling and penetrating agents such as glycerol, propylene glycol monoethyl ether, carbonates, hydrogen carbonates, guanidines, ureas and primary, secondary and tertiary phosphates; opacifiers such as latex, styrene/PVP and styrene/acrylamide copolymers; pearlescing agents such as ethylene glycol mono- and distearate and PEG-3 distearate; and blowing agents such as propane-butane mixtures, N₂O, dimethyl ether, CO₂ and air.

The selection of these other substances will be made by the specialist according to the desired properties of the agents. With regard to other optional components and the quantities of these components used, explicit reference is made to the relevant manuals known to the specialist. The additional active ingredients and auxiliary substances are preferably used in the preparations as contemplated herein in quantities of 0.0001 to 25 wt. % each, in particular 0.0005 to 15 wt. %, based on the total weight of the respective agent.

### Application of the Decolorizing Agent

In the process as contemplated herein, the previously described decolorizing agent is applied to applied to the dyed keratin material and rinsed off again after a contact time.

The application can be done with the (gloved) hand or with the help of an applicator, such as a brush or an applicate, or even a brush or a comb.

Depending on whether the user wants complete decolorization or only certain sections or areas. strands are to be decolorized, the decolorizing agent can be applied either to the entire keratinous material (such as the entire head of hair) or to specific parts or corresponding strands of the keratinous material or keratinous fibers.

After application, the decolorizing agent is left to act on the keratin material for a certain period of time. For example, an exposure time of 5 to 60 minutes, preferably of 5 to 30 minutes, further preferably of 5 to 15 minutes and most preferably of 5 to 10 minutes can be selected. After this exposure time, the decolorant is rinsed out again with water.

In a further preferred embodiment, a process as contemplated herein is wherein the decolorizing agent is applied to the colored keratin material and rinsed off again after an exposure time of 5 to 60 minutes, preferably of 5 to 30 minutes, further preferably of 5 to 15 minutes and very particularly preferably of 5 to 10 minutes.

The decolorizing agent can be applied to the keratin material at room temperature or at body temperature. However, the keratin material exposed to the decolorizing agent can also be exposed to elevated temperatures to support or coat the color fade. It is as contemplated herein if the decolorizing agent is applied to the dyed keratin material and the keratin material is heated to a temperature of 25 to 70° C., preferably 25 to 60° C., more preferably 30 to 55° C. and very particularly preferably 40 to 55° C. during the action of the decolorizing agent.

In the context of a further b embodiment, a method as contemplated herein is wherein
the decolorizing agent is applied to the dyed keratin material
the keratin material is heated during the action of the decolorizing agent to a temperature of from 25 to 70°, preferably from 25 to 60° C., more preferably from 30 to 55° C. and most preferably from 40 to 55° C., and then
the decolorizing agent is rinsed off again.

In addition to thermal support of the decolorization process, it is also possible to subject the keratin material exposed to the decolorizing agent to mechanical stress in order to improve the detachment of the film formed on the keratin material during coloring. For example, the keratin material can be massaged with the hands or combed with a comb or brush during the decolorization process. Any other mechanic stress suitable to improve the detachment of the colored film from the keratin material under the action of the decolorizing agent is also conceivable and encompassed by the process as contemplated herein.

In the context of a further preferred embodiment, a method as contemplated herein is wherein
the decolorizing agent is applied to the dyed keratin material,
the keratin material is combed, massaged, brushed or otherwise subjected to mechanical force during the action of the decolorizing agent, and then
the decolorizing agent is rinsed off again.

As previously described, the decolorizing agent as contemplated herein can be applied to decolorize keratin material that has been colored by application of a pigment, or at least one pigment and at least one amino silicone. If, for example, the user discovers after dyeing that the color result does not meet his requirements, he can take this as an opportunity to remove the dyeing again by applying the decolorizing agent.

Furthermore, the user can also plan a coloring and the subsequent decolorization from the outset, for example, if he wants to dye his hair for a particular occasion and then decolorize it again. For this purpose, the user can also be provided with all agents or formulations necessary for both coloring and decoloring.

Thus, a second object of the present disclosure is a method for coloring and later decolorizing human hair, comprising the following steps:

(1) Applying a colorant to the hair, the colorant comprising at least one amino-functionalized silicone polymer and at least one pigment, as already disclosed in detail in the description of the first subject matter of the invention,
(2) Allow the dye to act on the hair,
(3) Rinse the dye from the hair,
(4) Applying a decolorizing agent to the hair, the decolorizing agent having been disclosed in detail in the description of the first subject invention,
(5) Allowing the decolorizing agent to act on the hair,
(6) Rinse the decolorant out of the hair.

Thus, another object of the present disclosure is to provide a method for coloring and later decolorizing human hair, comprising the following steps:

(1) Applying a colorant to the hair, the colorant comprising at least one amino-functionalized silicone polymer and at least one pigment, as already disclosed in detail in the description of the first subject matter of the present disclosure,
(2) Allow the dye to act on the hair,
(3) Rinse the dye from the hair,
(4) Applying a decolorizing agent to the hair, the decolorizing agent having been disclosed in detail in the description of the first subject present disclosure,
(5) Allowing the decolorizing agent to act on the hair,
(6) Rinse the decolorant out of the hair.

The pigments and the amino-functionalized silicone polymers have already been disclosed in detail in the description of the first subject matter of the present disclosure. The decolorizing agent has also already been disclosed in detail in the description of the first subject matter of the present disclosure.

### Multi-Component Packaging Unit

It is particularly convenient for the user if the appropriate coloring and decolorizing agents are made available to him in the form of a multi-component packaging unit.

Thus, another counterpart of the present disclosure is a multi-component packaging unit (kit-of-parts) for dyeing and decolorizing keratin material, comprising separately packaged components:
a first container comprising a colorant, the colorant comprising at least one amino-functionalized silicone polymer and at least one pigment as already disclosed in detail in the description of the first subject matter of the present disclosure, and
a second container comprising a decolorizing agent, the decolorizing agent having been disclosed in detail in the description of the first subject present disclosure.
   Concerning the further preferred embodiments of the multicomponent packaging unit as contemplated herein and the use, mutatis mutandis what has been said about the process as contemplated herein applies.

### EXAMPLES

### 1. Formulations

The following formulations were prepared (all figures in wt. % o unless otherwise stated).

| Colorant (FM) | FM (wt. %) |
|---|---|
| Cetyl alcohol | 3.0 |
| Stearyl alcohol | 3.0 |
| Ceteareth-30, (Cetearyl alcohol, ethoxylated 30 EO) | 1.5 |
| Potassium dihydrogen phosphate | 0.35 |
| Disodium hydrogen phosphate | 0.72 |
| Unipure Red LC3071, organic pigment CI 15850 | 1.0 |
| 1.2-propanediol | 10.0 |
| Phenoxyethanol | 0.8 |
| Dow Coming 2-8566 (siloxanes and silicones, 3-[(2- | 1.0 |
| aminoethyl)amino]-2-methylpropyl Me, di-Me-siloxane. | |
| Sodium salicylate | 0.4 |
| Water | ad 100 |

| Decolorizer (EM) | EM-1 | EM-2 | EM-3 |
|---|---|---|---|
| (wt..-%) | Comparison | Comparison | Invention |
| Sodium laureth sulfate (C12-C14, | 8.7 | 7.0 | 14.0 |
| ethoxylated with 2 EO) | | | |
| Disodium cocoamphodiacetate | 2.0 | — | — |
| Laureth-6-carboxylate, sodium | — | — | — |
| salt | | | |
| Caustic soda/citric acid | ad pH 4.6 | ad pH 4.7 | ad pH 2.9 |
| Water | ad 100 | ad 100 | ad 100 |
| Decolorizer (EM) | EM-4 | EM-5 | EM-6 |
| (wt..-%) | Invention | Invention | Invention |
| Sodium laureth sulfate (C12-C14, | — | — | — |
| ethoxylated with 2 EO) | | | |
| Disodium cocoamphodiacetate | — | — | — |
| Laureth-6-carboxylate, sodium | 10.0 | 10.0 | 10.0 |
| salt | | | |
| Cocoamidopropylbetaine | — | — | — |
| Dimethylcocoylbetaine | — | — | — |
| Caustic soda/citric acid | ad pH 2.9 | ad pH 2.4 | ad pH 2.1 |
| Water | ad 100 | ad 100 | ad 100 |

### 2. Application

After preparation, the colorant (FM) was applied to hair strands (Kerling, Euronatural hair white). The dye was left to act for three minutes. Subsequently, the hair strand was washed thoroughly (1 minute) with water, dried and left to rest for 24 hours.

One of the dyed strands was measured with a colorimeter from Datacolor, type Spectraflash 450.

The decolorizer was applied to one colored strand of hair at a time, massaged in for 5 minutes and then rinsed with water. The hair decolorized in this way was allowed to dry and then colorimetrically measured.

The dE value used to assess color retention is derived from the L*a*b* colorimetric values measured on the respective strand part as follows:

dE=[(Lᵢ−L₀)²+(aᵢ−a₀)²+(bᵢ−b₀)]^{1/2}

L₀, a₀ and b₀=measured values of the undyed strand
Lᵢ, aᵢ and bᵢ=Measured values of the dyed/decolored strand

The smaller the dE value, the smaller the color distance compared to the undyed strand and the better the decolorizing effect.

| | | | | | Decolorizing |
|---|---|---|---|---|---|
| | L | a | b | dE | performance |
| Hair strand Euronatural | 73.18 | 2.35 | 22.09 | — | — |
| hair white, uncolored | | | | | |
| Dyeing with (FM) | 41.43 | 47.38 | 4.98 | — | — |
| Dyeing with (FM) and | 40.61 | 46.83 | 12.14 | 56.0 | bad |
| decolorization with (EM-1) | | | | | |
| Dyeing with (FM) and | 39.0 | 48.95 | 3.55 | 60.7 | bad |
| decolorization with (EM-2) | | | | | |
| Dyeing with (FM) and | 49.33 | 32.85 | 6.36 | 41.8 | medium |
| decolorization with (EM-3) | | | | | |
| Dyeing with (FM) and | 62.84 | 27.05 | 8.30 | 30.1 | good |
| decolorization with (EM-4) | | | | | |
| Dyeing with (FM) and | 57.29 | 27.45 | 6.84 | 33.4 | good |
| decolorization with (EM-5) | | | | | |
| Dyeing with (FM) and | 59.65 | 26.72 | 9.48 | 30.6 | good |
| decolorization with (EM-6) | | | | | |

dE = color distance to untreated hair

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A process for decolorizing keratinous material which has been colored by the application of at least one pigment, comprising
applying to the keratinous material a decolorizing agent comprising at least one anionic surfactant, and having a pH value of 1.0 to 3.5, and
rinsing the decolorization agent from the keratinous material after a contact time.

2. The process according to claim 1, wherein the decolorizing agent is applied to keratin material that has been colored by at least one amino-functionalized silicone polymer and at least one pigment.

3. The process according to claim 2, wherein the decolorizing agent is applied to keratin material that has been colored by at least one amino-functionalized silicone polymer having at least one secondary amino group.

4. The process according to claim 3 wherein the decolorizing agent is applied to keratin material that has been colored by at least one amino-functionalized silicone polymer comprising at least one structural unit of the formula (Si-Amino), 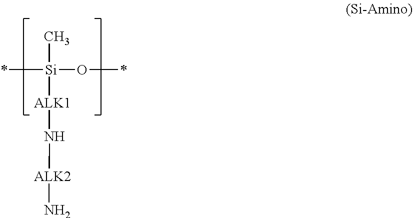
where
ALK1 and ALK2 independently represent a linear or branched C₁-C₂₀ divalent alkylene group.

5. The process according to claim 1 wherein the decolorizing agent is applied to keratin material colored by at least one amino-functionalized silicone polymer comprising structural units of formula (Si-I) and formula (Si-II)

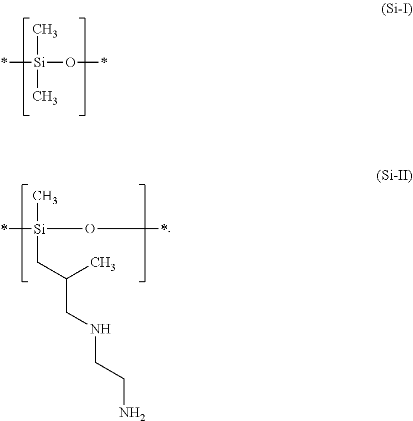

6. The process according to claim 1 wherein the decolorizing agent is applied to keratin material colored by at least one inorganic pigment selected from the group consisting of colored metal oxides, metal hydroxides, metal oxide hydrates, silicates, metal sulfides, complex metal cyanides, metal sulfates, bronze pigments and/or from colored mica- or mica-based pigments coated with at least one metal oxide and/or a metal oxychloride.

7. The process according to claim 1 wherein the decolorizing agent is applied to keratin material which has been colored by at least one organic pigment selected from the group consisting of carmine, quinacridone, phthalocyanine, sorghum, blue pigments having the color index numbers CI 42090, CI 69800, CI 69825, CI 73000, CI 74100, CI 74160, yellow pigments having the color index numbers CI 11680, CI 11710, CI 15985, CI 19140, CI 20040, CI 21100, CI 21108, CI 47000, CI 47005, green pigments with Color Index numbers CI 61565, CI 61570, CI 74260, orange pigments with Color Index numbers CI 11725, CI 15510, CI 45370, CI 71105, red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

8. The process according to claim 1, wherein the decolorizing agent comprises at least one anionic surfactant of the formula (T-1), 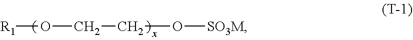
where
R₁ represents a linear or branched, saturated or unsaturated C₈-C₃₀ alkyl group,
x stands for an integer from 0 to 50, and
M is a hydrogen atom, ammonium (NH₄)₊ or an equivalent of a monovalent or polyvalent cation.

9. The process according to claim 1, wherein the decolorizing agent comprises at least one anionic surfactant of the formula (T-2), 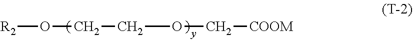
where
R2 represents a linear or branched, saturated or unsaturated C₈-C₃₀ alkyl group,
y stands for an integer from 0 to 16,
M is hydrogen or a metal such as an alkali metal, in particular sodium, potassium, lithium, an alkaline earth metal, in particular ½ magnesium, ½ calcium, ½ zinc, or an ammonium ion (NH4+).

10. The process according to claim 1 wherein the decolorant comprises—based on the total weight of the decolorant—one or more anionic surfactants in a total amount of 1.0 to 15.0 wt. %.

11. The process according to claim 10 wherein the decolorant has a pH of from 1.5 to 3.3.

12. The process according to claim 11 wherein the decolorizing agent is applied to the colored keratin material and rinsed off again after an exposure time of 5 to 60 minutes.

13. A process for dyeing and later decolorizing human hair, comprises the following steps:
(1) applying a colorant to the hair, the colorant comprising at least one amino-functionalized silicone polymer and at least one pigment,
(2) allowing the dye to act on the hair,
(3) rinsing the dye from the hair,
(4) applying a decolorizing agent to the hair, the decolorizing agent comprising at least one anionic surfactant and having a pH value of 1.0 to 3.5,
(5) allowing the decolorizing agent to act on the hair,
(6) rinsing the decolorizing agent out of the hair.

14. A multi-component packaging unit (kit-of-parts) for staining and decolorizing keratin material, comprising separately packaged:
a first container comprising a colorant, the colorant comprising at least one amino-functionalized silicone polymer and at least one pigment, and
a second container comprising a decolorizing agent, the decolorizing agent comprising at least one anionic surfactant and having a pH value of 1.0 to 3.5.

15. The process according to claim 1 wherein the decolorant comprises—based on the total weight of the decolorant—one or more anionic surfactants in a total amount of 6.0 to 11.0 wt. %.

16. The process according to claim 1 wherein the decolorant has a pH of from 2.0 to 3.0.
